(19) United States
(12) Patent Application Publication
(10) Pub. No.: US 2009/0324695 A1
(43) Pub. Date: Dec. 31, 2009

(54) BIOCOMPATIBLE POLYMER CERAMIC COMPOSITE MATRICES

(76) Inventors: Paul Ducheyne, Rosemont, PA (US); David I. Devore, Princeton, NJ (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/476,009

(22) Filed: Jun. 1, 2009

Related U.S. Application Data
(60) Provisional application No. 61/057,642, filed on May 30, 2008.

(51) Int. Cl. A61K 9/00; A61P 17/02

(52) U.S. Cl. 424/445

(57) ABSTRACT

Biocompatible composites of flexible polymers/copolymers films and dispersed solid silica-based ceramic microparticles are provided. The composites uniquely combine material and drug delivery properties that are essential for wound dressings and drug delivery applications. The flexible copolymer films are preferably tyrosine-based polycarbonates, and the ceramic microparticles are biodegradable silica-based glass particles that are preferably processed by a sol-gel methodology. The copolymers and the ceramic microparticles independently can contain therapeutic agents, are independently capable of binding these agents, and can independently release such agents. It is sufficient that either the polymer or the ceramic contains therapeutic agents, although both may contain them.

### CROSS-REFERENCE TO RELATED APPLICATIONS

This application claims the benefit of priority to U.S. Provisional App. No. 61/057,642, filed May 30, 2008, the entire contents of which are incorporated herein in their entirety.

### STATEMENT OF GOVERNMENT RIGHTS

Research leading to the disclosed inventions was funded, in part, by the United States Army, CDMRP grant number W81XWH-07-1-0438. Accordingly, the United States Government may have rights in the inventions described herein.

### FIELD OF TECHNOLOGY

The present invention is directed to drug “depots” or wound dressings which provide controlled delivery of therapeutic agents for healthcare applications.

### BACKGROUND

Early treatment of bodily wounds is generally limited to hemostasis and administration of pain medication. For example, for severe battlefield wounds, the initial treatment consists of applying hemostatic agents such as chitosan bandages and Quick-Clot™ zeolite. Wound dressings being deployed on the battlefield, however, are not designed to deliver pain medication. Existing injectable hydrogels, such as Durect's SABER™ system for delivery of bupivacaine, are not designed for battlefield applications because they cannot withstand the conditions that occur during transport of patients to medical facilities. Further, traditional anesthetic delivery systems such as direct injection, epidural catheters, and intra-articular indwelling catheters are not designed or convenient for battlefield applications. These modalities of local delivery of analgesics are not designed to withstand the conditions present during the transport of patients, have limited efficacy, have potential adverse clinical complications, and require highly trained medical personnel. As a result, on-the-field pain treatment of wounds is usually delivered in the form of systemic morphine injections, which have numerous unwanted and serious side effects.

Severe combat wounds, particularly blast wounds resulting from explosive devices, involve substantial tissue damage that produces sustained and often intense levels of pain throughout and beyond the early tissue healing process. If the pain is left untreated, the pain signals may be imprinted in the central nervous system, resulting in chronic pain. Continuous peripheral nerve block by local delivery of anesthetics immediately following trauma or surgical procedures has been suggested as having the potential to prevent chronic pain, including syndromes such as phantom limb pain. Thus, it is highly desirable to provide controlled delivery of local anesthetics directly to a wound.

In some instances, severe combat wounds, particularly blast wounds, also result in compartment syndrome. Compartment syndrome occurs when elevated intramuscular pressure decreases vascular perfusion of a muscle compartment to a point no longer sufficient to maintain viability of the muscle and neural tissue contained within the compartment. Compartment syndromes can result from multiple types of injuries including orthopedic (traumatic), vascular, iatrogenic, and soft tissue. Blast injuries now seem to fall in this category as well. In some cases the blast injury may only be part of soft tissue injury or it may be a combination of the other etiologies including components of orthopedic, vascular and/or soft tissue. More recently with the increasing number of casualties from blast injury, it is hypothesized that the blast causes a direct injury to the muscle that results in swelling and a secondary compartment syndrome.

Generally, in compartment syndromes, there is an increasing pressure within a tissue compartment that needs to be released as soon as possible, often within 4 to 6 hours. Compartment syndromes must be treated early in the time line of wound care that begins at the battlefield and ends in the hospital. If a compartment syndrome is not diagnosed early, a Volkmann contracture may occur with massive loss of all tissues within the compartment. Untreated compartment syndrome can lead to tissue necrosis, permanent functional impairment, renal failure, and death. However, the standard diagnosis of compartment syndrome by clinical signs—including myoneural pain with passive stretch, paresthesia, and paresis—is often masked by other injuries in patients with blast injuries who suffer polytrauma.

The treatment of compartment syndrome requires the release of the fascia that enclose the compartments within the first three to six hours to prevent irreversible injury to the nerves and muscles. Once the compartments are released the open wounds are treated with dressings to prevent infection and protect the wound. In some cases, a specialized V.A.C. (Vacuum Assisted Closure System) is used to cover and protect the wound. The open wounds are then kept dressed for 48 to 72 hours until the patients are returned to the operating room for a second look to allow further debridement of non-viable muscle tissue if indicated. Fasciotomies, however, extends hospital stays and changes a closed injury to an open injury, greatly increasing the chance of infection. Further, there is some debate about the criterion for performing a fasciotomy, with recommendations varying from prophylactic fasciotomy at normal pressure to finding a pressure from 30 mm Hg to 45 mm Hg.

It has been suggested that impeding the early cellular events leading to ischemia and pressure build up in the compartment may be the first line of defense. Thus, it would be desirable to provide controlled delivery of therapeutic agents to prevent the late-stage problems of compartment syndrome and initiate regeneration of healthy tissue.

There remains a great need for materials for the treatment of wounds that effect the controlled release of pharmaceutically active molecules. Controlled release focuses on delivering biologically active agents locally over extended time periods (Heller, J., “Use of polymers in controlled release of active agents”, Controlled Drug Delivery: Fundamentals and Applications, Robinson, Jr, et al., editors, New York, Dekker, 1987; Radin, S, Ducheyne, P., “Nanostructural control of implantable xerogels for the controlled release of biomolecules”, Learning from Nature How to Design New Implantable Materials: from Biomineralization Fundamentals to Biomimetic Materials and Processing Routes, Reis, R. L., and Weiner, S, editors, New York, Kluwer, 2005). The site specificity of the delivery reduces the potential side effects that can be associated with general administration of drugs through oral or parenteral therapy (Radin, S., ibid.; Kortesuo, P. et al., J. Control. Release 2001; 76(3):227-238). Prevalent mechanisms for the delivery of biological agents by controlled release devices are either resorption of the drug carrier material or diffusion. The resorption of these devices may, however, cause an inflammatory tissue response which interferes with the treatment sought for with the biomolecules (Ibim, S. M., et al., Poly(anhydride-co-imides): In vivo biocompatibility in a rat model, Biomat., 1998; 19:941-951).

### SUMMARY

The present invention is directed to drug depots or wound dressings comprising biocompatible composites of flexible polymers and dispersed solid silica-based ceramic microparticles adapted to bind and release therapeutic agents, thereby providing controlled delivery of the therapeutic agents for healthcare applications. The particles can have a wide variety of shapes, including short fiber shapes, long strand-like fiber forms, and others. The ceramic microparticles are preferably biodegradable silica-based glass particles. A preferred route of processing these particles is by a sol-gel methodology, although other methods may be utile. The polymers are preferably biocompatible hydrogels, including those known to be readily biodegradable, such as: poly(vinyl alcohol); poly(ethylene oxide) (PEO, or PEG); copolymers of PEO or PEG with poly(lactic acid) (PLA), polyglycolic acid (PGA), copolymers of lactic and glycolic acid (PLGA), polysaccharides, poly(desaminotryosyl tyrosine ester) or poly(desaminotyrosyl tyrosine carbonate). The polymers and copolymers may be cross-linked, either by covalent or ionic bonding, to promote critical performance properties including gelling, fluid adsorption, and increased mechanical strength.

These composites provide control of binding and release of therapeutic agents, thereby providing controlled delivery of the therapeutic agents for healthcare applications. The polymers and the ceramic microparticles independently can contain therapeutic agents, are independently capable of binding these agents, and can independently release such agents. It is sufficient that either the polymer or the ceramic contains therapeutic agents, although both may contain them. The composite of the ceramic in the polymer provides a unique matrix that enables better control of the kinetics of delivery of the therapeutic agents than can be attained by either the polymers or sol-gels alone. Embedding ceramics particles in a polymeric film also enables to use the outstanding release properties of the particles in applications where a solid sheet is needed for treatment, such as in wound dressings. The composite is useful in depot delivery of therapeutic agents such as organic drug compounds, genes, oligonucleotides, and proteins, and in wound treatment applications such as for compartment syndrome, chronic and phantom pain treatment, hemostasis, infection control, and otherwise. Thus a wide variety of therapeutic agents such as antibiotics, analgesics, vasodiolators, and vasoconstrictors may be so delivered. The release of one or more therapeutic agents from the present composites may be pseudo first order release.

The efficacy of prior controlled delivery devices for therapeutic agents is generally limited by the problem of so-called burst release kinetics. The composites of this invention reduce or eliminate the burst release, instead providing continuous and constant rates of release of the therapeutic agent which are essential for sustained, effective therapeutic activity. The composites uniquely combine material and drug delivery properties that are essential for wound dressings and drug delivery applications. The composites are biocompatible (i.e. substantially non-cytotoxic and non-inflammatory), biodegradable, flexible, mechanically robust, and capable of providing continuous controlled release of a wide array of therapeutic agents for a useful period of time. Further, the robust, flexible nature of the composite enables their use as implanted depots or wound dressings not only in hospitals and civilian uses but also in the far more demanding conditions of military uses such as on a battlefield or field hospital.

In accordance with one aspect of the invention, biodegradable, biocompatible ceramic-polymeric flexible composite films provide controlled delivery of local anesthetics directly to the wound site to provide pain relief. The biomaterial composite films provide sustained treatment of the peripheral nerves located at the wound site with a local anesthetic that functions as a sodium channel blocker to shut down the firing of the afferent axons that carry the pain signals back to the brain. This technology can reduce or eliminate the imprinting process in the central nervous system that is recognized as a key component of chronic pain. Further, delivery of pain medication by the robust biomaterial composites, beginning on the battlefield or in combat support hospitals or in surgical procedures at veterans and civilian hospitals, may lead to reduced morbidity, decreased postoperative narcotic usage, and the attenuation of chronic pain syndromes.

In accordance with another aspect of the invention, provided is a biocompatible composite designed to counteract the effects of compartment syndrome of the tissues. Thus, the present invention provides composites of biocompatible tyrosine-based block copolymers and bioresorbable silicon-based ceramic sol-gels that deliver anti-apoptotic and pro-angiogenic factors, seal damaged cell membranes to repair damaged tissues, and absorb extracellular fluid within the compartment to reduce the hydrostatic pressure and minimize the extent of damaged tissue. These treatments may act prophylactically and thereby reduce, if not eliminate the need for fasciotomies. Furthermore, some of these treatments may accelerate healing after fasciotomies.

### BRIEF DESCRIPTION OF THE DRAWINGS

FIG. 1 illustrates the release pattern of bupivacaine from HCl-catalyzed xerogel granules for different bupivacaine loads (mg of bupivacaine per gram of dry weight sol).

FIG. 2 illustrates the cumulative release of bupivacaine from acid-base catalyzed microspheres.

FIG. 3 shows the chemical equation for the polymerization of tyrosine-derived diphenolic monomers with blocks of poly(ethylene glycol) (PEG), which produces a new class of elastomeric poly(ether carbonate)s.

FIG. 4 depicts bupivacaine release kinetics from each of xerogels, polymer-drug complexes, and composites in accordance with the present invention.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

In the present disclosure the singular forms “a,” “an,” and “the” include the plural reference, and reference to a particular numerical value includes at least that particular value, unless the context clearly indicates otherwise. Thus, for example, a reference to “a polymer” is a reference to one or more of such materials and equivalents thereof known to those skilled in the art, and so forth. When values are expressed as approximations, by use of the antecedent “about,” it will be understood that the particular value forms another embodiment. As used herein, “about X” (where X is a numerical value) refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable.

The disclosures of each patent, patent application, and publication cited or described in this document are hereby incorporated herein by reference, in their entirety.

Biocompatible composites of flexible polymers/copolymers and dispersed solid silica-based ceramic microparticles are provided. The composites uniquely combine material and drug delivery properties that are essential for wound dressings and drug delivery applications: biocompatibility (non-cytotoxic and non-inflammatory), biodegradability, flexibility, mechanical robustness, and continuous controlled release of a wide array of therapeutic agents.

The polymers/copolymers are biocompatible hydrogels, including for example, those known to be readily or functionally biodegradable, such as: poly(vinyl alcohol); poly(ethylene oxide) (PEO, or PEG); copolymers of PEO or PEG with poly(lactic acid) (PLA), polyglycolic acid (PGA), copolymers of lactic and glycolic acid (PLGA), polysaccharides, poly(desaminotryosyl tyrosine ester) or poly(desaminotyrosyl tyrosine carbonate). The polymers/copolymers can be prepared as solvent-cast or compression molded films.

The ceramic particles can have a wide variety of shapes, especially including short-fiber shapes, long strand-like fiber forms, nearly spherical particles, and irregular shapes. The ceramic microparticles are biodegradable silica-based glass particles. These ceramic microparticles are preferably processed by a sol-gel methodology, although this is not necessary. The sol-gels may be synthesized as monoliths or as 10 micrometer granules.

The polymers/copolymers may be cross-linked, either by covalent or ionic bonding, to promote critical performance properties including gelling, fluid adsorption, and increased mechanical strength. Crosslinking and substantially crosslinking moieties are known per se to polymer scientists. Embedding ceramics particles in a polymeric film also enables to use the outstanding release properties of the particles in applications where a solid sheet is needed for treatment, such as in wound dressings.

The polymers/copolymers and the ceramic microparticles independently can contain therapeutic agents, are independently capable of binding these agents, and can independently release such agents. It is sufficient that either the polymer or the ceramic contains therapeutic agents, although both may contain them. The composite of the ceramic in the polymer provides a unique matrix that enables far better control of the kinetics of delivery of the therapeutic agents than can be attained by either the polymers or sol-gels alone. These composites provide unique control of binding and release of therapeutic agents, thereby providing controlled delivery of the therapeutic agents for healthcare applications. The composites combine the advantages of the drug binding and release kinetics of silica sol-gels with the mechanical flexibility and drug binding of polycarbonate films. The drug delivery system of the present invention permits fine tuning of drug loading and drug release kinetics while providing the mechanical strength and stability properties characteristic of heterogeneous composites. The composites of this invention are designed to reduce burst release and provide the continuous and constant rates of release of a therapeutic agent that is essential for sustained, effective therapeutic activity. The release of one or more therapeutic agents from the present composites may be pseudo first order release (i.e., the release kinetics of the present composites may be characterized by a substantially constant release of therapeutic agent over time).

Conditions for synthesizing sol-gel powders may be controlled to produce a particular controlled release profile for a therapeutic agent corresponding to a concentration with known therapeutic effect. The parameters that may be varied are the method of making powder (either pellet casting and grinding, or microsphere synthesis by emulsifying the sol-gels), powder size, de-ionized water-to-tetraethoxysilane ratio, and molecule concentration. The drug molecules, incorporated in nano-sized pore channels of the sol-gels and non-covalently bound by the copolymers of the biocompatible film, will release by diffusion through the aqueous phase that penetrates into the composite films. The sol-gels can be synthesized as monoliths or as 10 micrometer granules and the copolymers can be prepared as solvent-cast or compression molded films.

Process parameters such as de-ionized water to tetraethoxysilane ratio, molecule concentrations, powder size, and particle formation process may be optimized for maximum loading efficiency of each drug. Also, each of the parameters of the sol-gel synthesis affects the fundamental properties of the particles that control release of the therapeutic agent. These parameters include specific surface area, granule or powder size, and pore size and porosity. Formation of composite films of the sol-gel microparticles in polymers, such as in poly(DTR-co-PEG carbonate), may be by compression molding; the copolymer compositions (pendent ester R chain lengths, PEG molecular weight and PEG/DTR molar ratios) may be varied systematically to achieve an optimum loading efficiency of the drug-loaded silica sol-gel microparticles and to improve the mechanical properties of the films, such as tensile and flex strengths.

The composite of the present invention is useful in depot delivery of therapeutic agents such as organic drug compounds, genes, oligonucleotides, and proteins, and in wound treatment applications such as for compartment syndrome, chronic and phantom pain treatment, hemostasis, and infection control. The composites of the present invention may be useful in various therapeutic applications, including treatment of pain resulting from wounds and prophylactic treatment of compartment syndrome associated with wounds. For the treatment of pain, silica-based sol-gels and tyrosine-based copolymers may be synthesized to effectively bind and release therapeutic agents such as bupivacaine and mepivacaine. For the prophylactic treatment of compartment syndrome, sol-gels and copolymers may be synthesized to effectively bind and release anti-apoptotic and pro-angiogenic factors. While the therapeutic composites of the present invention may be described in connection with a single drug, it will be understood by those skilled in the art that the therapeutic composites are capable of concurrent delivery of multiple drugs.

### Pain Treatment

Provided is a novel approach to the treatment of chronic pain arising from wounds with severe tissue damage and/or from surgical procedures. This approach entails controlled release of a selected local anesthetic from biocompatible composite films directly to the wound site beginning as soon as possible after the wound or surgery occurs. The biocompatible composite films provide sustained treatment of the peripheral nerves located at the wound site with a local anesthetic that functions as a sodium channel blocker to shut down the firing of the afferent axons that carry the pain signals back to the brain. This technology can potentially reduce or eliminate the imprinting process in the central nervous system that is recognized as a key component of chronic pain.

In accordance with this aspect of the invention, a local anesthetic may be bound to a composite matrix comprised of silica sol-gel microparticles incorporated in a tyrosine based polycarbonate-PEG film to provide controlled release of the anesthetic. The local anesthetic is preferably mepivicaine or bupivicaine, because of their high activity with low cardiovascular side effects. The composite films are preferably effective for up to 72 hours, permitting easy use on the battlefield, in combat support hospitals, and civilian and veterans' hospitals. Bupivacaine and mepivacaine may be incorporated in silica sol-gel powders, either by casting pellets and grinding the sol-gels to granules or by making microspheres of the same size range. The immediate and sustained delivery of local anesthetic will enable quicker recovery times, shorter hospital stays, earlier achievement of physical therapy milestones, and lower rates of narcotic use and abuse among military and civilian patient populations.

### Prophylactic Treatment Of Compartment Syndrome

In compartment syndromes, there is a zone of tissue that is between normal and irreversibly damaged, and in this zone anti-apoptotic and pro-angiogenic factors may be useful to restore function. Thus, in accordance with one aspect of the invention, provided is a prophylactic treatment of a wound site to avoid the onset of compartment syndrome and associated fasciotomy treatment. Even when fasciotomy is ultimately required, treatment in accordance with the invention provides for more rapid and complete healing of incision and wound sites.

In acute compartment syndrome, fluid accumulates and the intramuscular pressure (IMP) increases. Removal of only about 1 ml of interstitial fluid may result in a reduction of intramuscular pressure such that intramuscular pressure (IMP) is restored to a normal range. Thus, in accordance with one aspect of the invention, composites made from polymers such as tyrosine-based block copolymers and silicon-based ceramic sol-gels may be designed as a polymer-ceramic “superslurper”-type hydrogel wound dressing to remove fluid from injured muscle compartments. The biocompatible composites may be composed of tyrosine-based copolymers and silica sol-gels in the form of physically blended composite films that are adapted to absorb 100% or more of their weight in body fluid while maintaining their flexibility, adhesion, and mechanical integrity. To provide “superslurper”-type fluid adsorption, well-established synthetic polymer chemistry methods for forming cross-linked polymers may be employed.

Further, the composite dressing is capable of concurrently delivering a selected therapeutic agent to the wound site. The therapeutic agent may be incorporated in resorbable microspheres of ceramic sol-gel that are embedded in the copolymer hydrogel film. The therapeutic agent incorporated into the sol-gel powder may include one or more of an anti-apoptotic factor, a pro-angiogenic factor, and a polymeric surfactant. Thus, a copolymer hydrogel film may be prepared by physically mixing the sol-gel powders and the copolymers prior to molding the composite hydrogel and may be provided to a clinician as a flexible, adherent film wound dressing that is biodegradable.

### Tyrosine-Based Polycarbonate-Poly(Ethylene Glycol) Copolymers

Degradable polyesters, poly(glycolic acid) (PGA), poly(lactic acid) (PLA), their copolymers (PLGA), and polydioxanone, are the predominant synthetic, degradable polymers with extensive regulatory approval histories in the USA. Although the utility of these materials as sutures and in a number of drug delivery applications is well established, these polymers cannot meet many of the material properties required for drug delivery devices. For example, all of these polyesters release acidic degradation products, limiting their utility to applications where acidity at the implant site is not a concern. They also tend to be relatively rigid, inflexible materials, a disadvantage when mechanical compliance with soft tissue or blood vessels is required. Finally, the chemical properties of these polyesters is not substantially tunable, being limited to only a few combinations of fixed monomer structures, which limits thermodynamic and kinetic parameters that control drug binding and release.

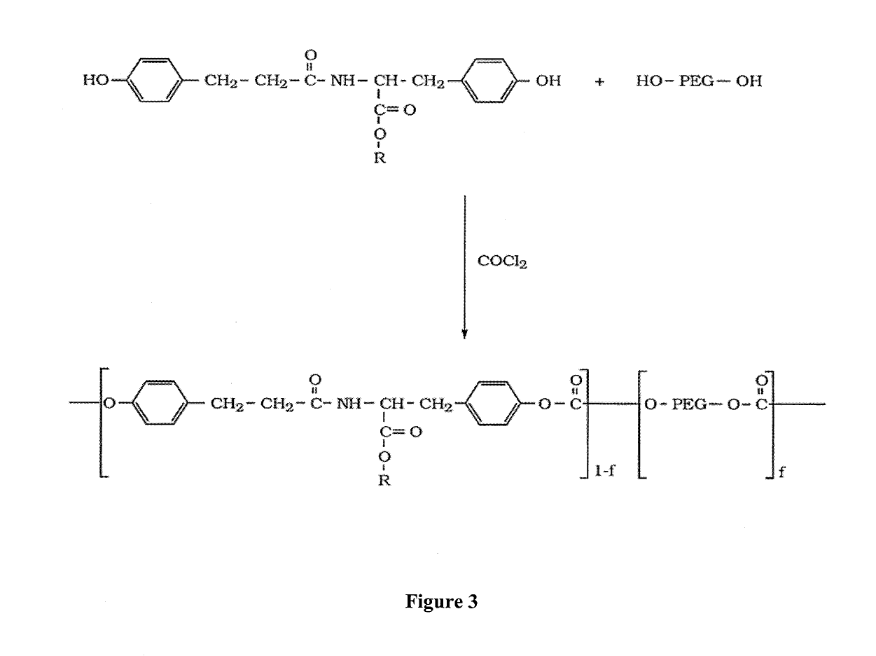

Thus, provided in the present invention is a flexible, biocompatible film that forms a stable composite with the silica sol-gels. The present invention encompasses a broad class of tunable, desaminotyrosyl tyrosine ester (DTR) diphenolic monomers that can be used to prepare polycarbonates and other polymer families. Among these polymers, tyrosine-derived polycarbonates have been studied most extensively and have been found to be tissue-compatible, strong, tough, hydrophobic materials that degrade slowly under physiological conditions (cf, J. M Pachence and J. Kohn, Biodegradable Polymers, in Principles of Tissue Engineering, 2nd Ed., R. P. Lanza, R. Langer, J. Vacanti (eds.), Academic Press, San Diego, 2000, pp. 272-273). Further, it is preferable to use tyrosine-based block copolymers rather than polylactides because of the far greater tunability of the tyrosine-based blocks and because the polylactides are known to have inflammatory effects in vivo whereas the tyrosine-based copolymers do not. When these tyrosine-derived diphenolic monomers are copolymerized with blocks of poly(ethylene glycol) (PEG), a new class of poly(ether carbonate)s is obtained that is elastomeric with remarkable tensile strengths and elongations, as shown in FIG. 3.

### Synthesis of Poly(DTR-co-PEG Carbonate)

These copolymers are referred to as poly(DTR-co-fPEG M carbonate) where R represents the type of ester pendent chain, f represents the percent molar fraction of PEG units present within the backbone, and M represents the molecular weight of the PEG blocks. Thus, poly(DTE-co-5% PEG1000 carbonate) refers to a copolymer prepared from the ethyl ester of desaminotyrosyl-tyrosine containing 5 mol % of PEG blocks of average molecular weight of 1000 g/mol. This molecular design provides tunability through three independent variables to enable optimization of materials properties (i) the pendent chain R, (ii) overall PEG content f, and (iii) length (molecular weight) M of the PEG block.

There are an enormous number of possible structures with this molecular design, including copolymers of poly(DTO-co-f PEG1000 carbonate), where f=0%, 10%, 40% and 70% to provide a range of hydrophobic-to-hydrophilic properties. DTO (i.e, the octyl ester) is selected because it has been identified as the ester having the optimal thermodynamic solubility parameter for binding hydrophobic drug molecules. Synthesis is performed by adding the DTO monomer and PEG to round bottom flasks containing methylene chloride and anhydrous pyridine. At room temperature, phosgene solution in toluene is added over 90 min to the reaction mixture with overhead stirring. Tetrahydrofuran (THF) is then added to dilute the reaction mixture to a 5% (w/v) solution. The copolymer is precipitated by slowly adding the mixture into 10 volumes of ethyl ether. For further purification, copolymers with lower PEG content (<70% by weight) are redissolved in THF (5% w/v) and reprecipitated by slowly adding the polymer solution into 10 volumes of water. Copolymers with higher PEG content (70% by weight) are redissolved in THF (10% w/v) and reprecipitated by slowly adding the polymer solution into 10 volumes of isopropanol. In each case, the precipitated copolymer is collected and dried under vacuum.

The molecular weight of the copolymers may be controlled by the duration of the reaction and determined by gel permeation chromatography using THF as the solvent and using polystyrene standards. Chemical structure and polymer purity may be monitored by FT-IR, H-NMR, and C-NMR. The glass transition temperatures (T_{g}), crystallinity, and melting points of each copolymer may be determined by differential scanning calorimetry (DSC) and the decomposition temperature obtained by thermogravimetric analysis (TGA), with heating rates for both DSC and TGA of 10° C./min using an average sample size of 15 mg.

Polycarbonate copolymers of poly(ethylene glycol) (PEG) and desaminotyrosyl tyrosine esters (DTR) may be prepared by solution phosgenation as illustrated in FIG. 3. These copolymers have weight-average molecular weights up to about 200,000 and have symmetrical molecular weight distributions. To obtain structure-activity relationships, copolymers are prepared with either 5% PEG1000 or 5% PEG2000 and different pendent ester chains (R=E (ethyl), B (butyl), H (hexyl), and O (octyl)). Also, the effect of PEG content is determined by preparing a series of poly(DTE-co-PEG1000 carbonate)'s with PEG content ranging from 1 mol % to 70 mol %. All of these copolymers are soluble in common organic solvents and those with high PEG content (70 wt %) are also soluble in water. Increasing the length of the hydrophobic pendent R chain lowers the glass transition temperature, T_{g}, in a linear fashion. The copolymers are thermally stable up to about 300° C.

Thin composite films of the silica sol-gels and poly(DTO-co-PEG carbonate) copolymers may be prepared by compression molding. Films may be prepared by physically mixing the sol-gel powders and the copolymers prior to molding. The weight ratio of the sol-gels to copolymers from 5% to 95% may be varied to control the mechanical and drug release properties of the resultant composites. The processing temperature may be set at 30-35° C. above the glass transition temperature, T_{g}, of the copolymers. To minimize polymer adhesion to the metal plates of the mold, two Teflon sheets may be added between the polymer and metal plates. The mechanical properties of the thin (approx. 0.1 mm) compression molded composite films may be tested on a Sintech 5/D tensile tester according to ASTM standard D882-91 at room temperature. For each composite film, four individual specimens may be used to obtain a reliable calculation of the elastic modulus and the yield point may be determined based on the zero slope criterion. The modulus, strength, and elongation may be obtained from stress-strain curves and averaged from five separate runs. The thickness of the composite films may be adjusted if necessary to provide for higher total quantities of drug release.

### Properties of Tyrosine Based Polycarbonates

The effect of PEG in the backbone of the tyrosine derived polycarbonates may be determined in compression molded samples that are subjected to mechanical analysis both in the dry state and the wet state. The copolymers with low PEG levels are strong, tough and have high tensile stiffness and strength. As the PEG content is increased, the polymers lose their stiffness and strength. Copolymers containing more than 5 mol % PEG are flexible, soft elastomers in the wet state.

The binding and release of organic drug compounds by the copolymers is a function of the hydrophobicity of the drug molecules as well as the hydrophobicity of the copolymer. The relative affinity of the copolymers for a drug can be predicted by their thermodynamic solubility parameters.

### Silica Sol-Gel Controlled Release Materials

Previously, it has been known to prepare certain bulk sol-gel materials for use in therapeutic regimes. The preparation of sol-gels generally as well as sol-gels having pharmaceutically active species in them has been disclosed in a number of U.S. patents, including several patents to one of the inventors of this invention. These include U.S. Pat. Nos. 5,874,109; 5,849,331; 5,817,327; 5,861,176; 5,871,777; 5,591,453; 5,830,480; 5,964,807; and 6,569,442. Each of these is incorporated herein by reference in order to set forth a number of ways of preparing sol-gels generally useful to the present invention, especially certain sol-gels having pharmaceuticals included within them.

Silica-based ceramic sol-gel technology provides tunable porosities capable of controlled delivery of a broad range of hydrophilic and hydrophobic therapeutic agents such as growth factors, anti-oxidants and antibiotics. The sol-gels can be prepared in various physical forms, including pellets, thin films or powders, in controlled sizes of 1 μm and larger. Organosilanes such as tetraethyoxysilane (TEOS) or tetramethoxysilane (TMOS) are used as the precursor molecules for the synthesis of the sol-gels via hydrolysis and condensation reactions. The hydrolysis reaction, which can be either acid or base catalyzed, replaces alkoxide groups with hydroxyl groups. Siloxane bonds (Si—O—Si) are formed during subsequent condensation. Alcohol and water are byproducts of the condensation reaction and evaporate during drying. Theoretically, the overall reaction is as follows:

n Si(OR)4+2n H2 O→n SiO2+4n ROH

However, in reality, the completion of the reaction and the chemical composition of the resulting product depend on the excess of water above the stoichiometric H₂O/Si ratio of 2. A number of other sol-gel processing parameters (such as pH of the sol, type and concentration of solvents, temperature, aging and drying schedules, etc.) can also affect the composition, structure, and properties of the resulting product.

Drug molecules are incorporated in the nano-sized pore channels and are released by diffusion through the aqueous phase that penetrates into these pores. The variation of processing parameters leads to variations of pore size (in the nanometer range) and porosity, which in turn affect the release rates of the drug molecules. Using a room temperature processing method, drug molecules are incorporated in nano-sized pore channels of the sol-gels and are released by diffusion through the aqueous phase that penetrates into these pores. The conditions for synthesizing sol gel powder may be adjusted to produce sol-gel powder having a release profile (amount released per weight of powder) for a selected drug molecule, which corresponds to a concentration with known therapeutic effect. The parameters that may be adjusted are the method of making powder (e.g. either pellet casting and grinding, or microsphere synthesis by emulsifying the sol gels), powder size, de-ionized water to tetraethoxysilane ratio (a parameter associated with the initial solution from which the sol gels are made), and molecule concentration. Thus, for a given drug molecule, the size, surface character, nanostructural pore size, and porosity of the sol-gel powder may be controlled to provide a desired release concentration of the drug molecule.

### Synthesis of Bupivacaine Containing Acid Catalyzed Xerogel

Acid catalyzed xerogel granules containing bupivacaine may be synthesized as follows. 10 ml of Tetraethoxysilane, TEOS [Si(OC₂H₅)₄, Strem] is used as a silica precursor. TEOS, de-ionized water (DI water/TEOS molar ratio 6:1), and 1 N HCl (0.25 or 0.3 ml) are mixed by using magnetic stirring at 1,100 rpm. Stirring is continued until a one-phase solution (sol) is formed. Stirring speed is then reduced to mid speed (e.g. 660 rpm). The sol is stirred for 30 more minutes. Bupivacaine incorporation is achieved by adding either aqueous or methanol solutions of bupivacaine: 40 mg of bupivacaine per ml of de-ionized water or 70 mg of bupivacaine per ml of methanol. Bupivacaine-water solutions are added to the mixture prior to the sol formation; whereas, bupivacaine-methanol solutions are added after. Acid catalyzed TEOS sol is also synthesized with 0.3 ml of 1 N HNO₃. HNO₃ catalyzed sol-gels are synthesized at water to TEOS molar ratio of 6 and theoretical bupivacaine loading of 50 mg/g. Time to one-phase solution is 30 minutes and 20 minutes for 0.25 and 0.3 ml 1 N HCl, respectively.

### Synthesis of Bupivacaine Containing Acid-Base Catalyzed Xerogels

To synthesize acid-base catalyzed xerogels, 10 ml of TEOS is mixed with de-ionized water to obtain a water to TEOS molar ratio of 6, and is hydrolyzed by 0.1 M HCl. After formation of one-phase solutions, bupivacaine dissolved in methanol is added (70 mg of bupivacaine/ml of methanol). Sols are synthesized at theoretical loadings of 50 mg/g and 30 mg/g. Stirring speed is reduced and allowed to mix for 15 minutes. The beaker is then placed in an ice-bath for 10 minutes. The beaker and ice-bath are placed back onto the stirrer and 2.4 ml of the alkaline solution 0.08 M NH₄OH is added dropwise to the sol.

### Synthesis of Bupivacaine Containing Silica Derived Microspheres

Formation of the sol-gel is followed by an emulsification to form microspheres. Microsphere synthesis includes various steps: formation of an acid-catalyzed sol (2.4 ml of 0.1 M HCl); addition of bupivacaine-methanol solution; addition of 2.6 ml base and 0.08 M NH₄OH; checking the time to gelation (less than 30 min); dropwise addition of the sol-gel mixture into a beaker of 100-ml vegetable oil that is moved at high speed (spun around).

The volume of alkaline solution that is added is varied in order to maintain the pH of the sol-gel mixture in its optimal range, such that the time to gelation is long enough for the sol-gel to form an emulsion in oil. After emulsification, the microspheres precipitate to the bottom of the beaker. After addition of de-ionized water to the beaker, the oily layer is poured off and the microspheres are collected and rinsed with ethanol. The particles are then filtered though 70 μm nylon microporous filters.

### In Vitro Release Study—Experimental Parameters

Release studies of bupivacaine from sol-gel particles (ground granules or microspheres) were conducted in phosphate buffered saline (PBS, pH 7.4). 25 mg of the particles were immersed in 5 ml PBS and then incubated at 37° C. while shaken at 100 rpm. The solutions were exchanged daily. Concentration of released bupivacaine was measured spectrophotometrically at 265 nm.

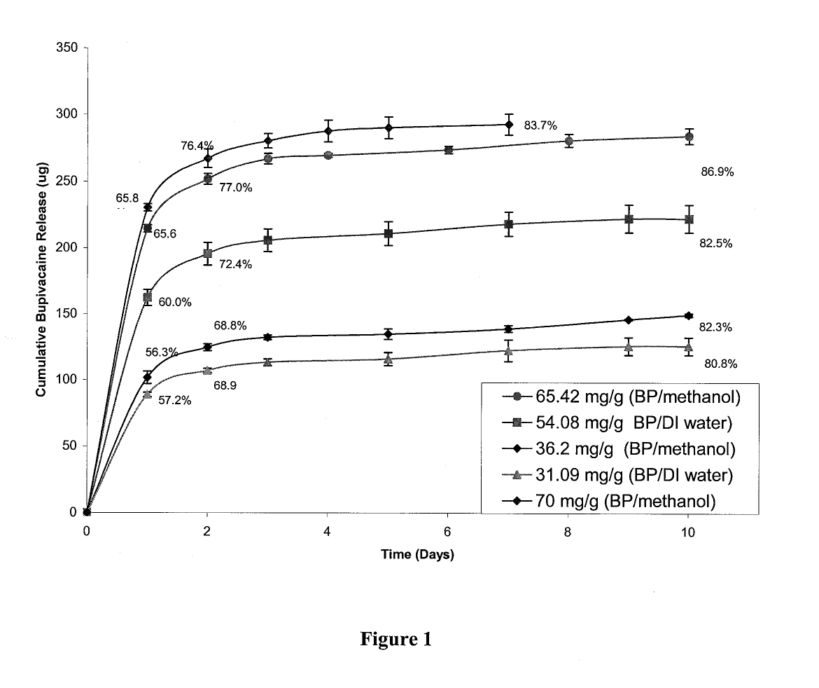

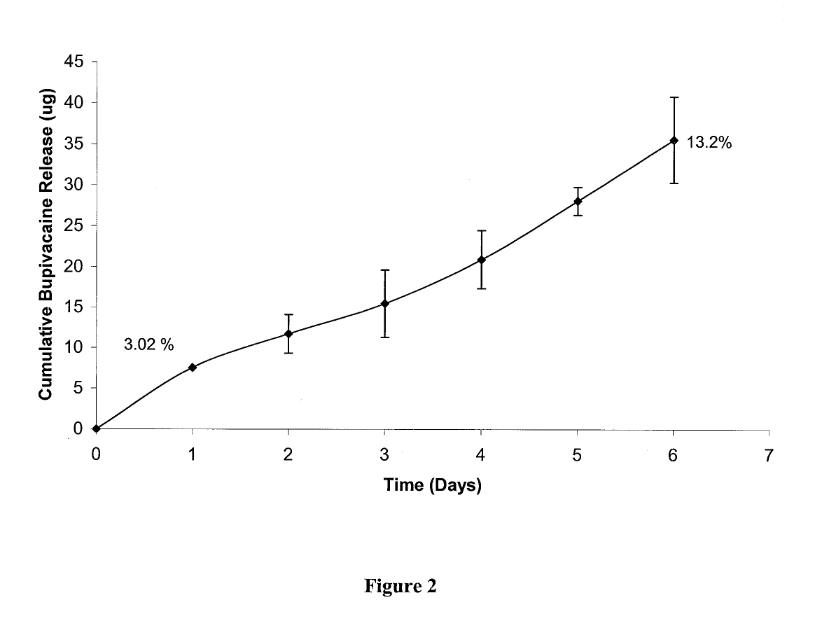

FIGS. 1 and 2 show release data from granules (cast and ground sol-gels) and microspheres (emulsified sol-gels) respectively. FIG. 1 shows the release pattern of bupivacaine from HCl-catalyzed xerogel granules. Each series has a different bupivacaine load (mg of bupivacaine per gram of dry weight sol) and was synthesized with bupivacaine/methanol solution or bupivacaine/de-ionized water solution. For these HCl-catalyzed xerogel granules, R=6, granules size 210-500 μm, 5 mg of dw sol/ml of PBS. The error bars signify one standard of deviation (n=3). FIG. 2, shows the cumulative release of bupivacaine from acid-base catalyzed microspheres. The microspheres contain 50 mg of bupivacaine per gram of SiO2. They were immersed in PBS (5 mg of dry weight sol/ml of PBS). For these acid-base catalyzed microspheres, R=6 and emulsification speed=330 rpm. The error bars signify one standard of deviation (n=3). Due to time constraints, the results for this immersion study are incomplete. Differences in release properties arise from differences in surface properties and pore properties of the particles.

### Release Kinetics

A study was conducted to ascertain the respective in vitro release kinetics of (1) bupivacaine from tyrosine-PEG-derived poly(ether carbonate) copolymers, (2) sol gel ceramic granules, and (3) polymer-ceramic composite matrices in accordance with the present invention.

Approximately 30 mg of samples (xerogels, copolymers, and composite films) were incubated in 6 mL PBS at 37° C. and 100 rpm using a Julabo SW2 water bath shaker. Periodically, the incubation medium was completely withdrawn and replaced with 6 mL fresh buffer. The withdrawn samples were diluted 1:1 (v/v) with acetonitrile and analyzed by HPLC. All experiments were performed in triplicate. The bupivacaine concentrations were assayed by high performance liquid chromatography (HPLC) using a Waters 2695 HPLC system equipped with a Waters 2489 UV/V is detector that was set at 210 nm for bupivacaine detection. Chromatographic separations were achieved using a Perkin-Elmer Pecoshere HS-3 C18 reversed-phase column, 3 μm particle size, 33×4.6 mm, at 25° C. Standard calibration curves were prepared at concentration ranging from 0.97 μg/mL to 0.25 mg/ml and exhibit linear behavior over this range of concentration. The detection limit was 0.23 μg, as determined by the standard deviation of the response and the slope of the calibration curve.

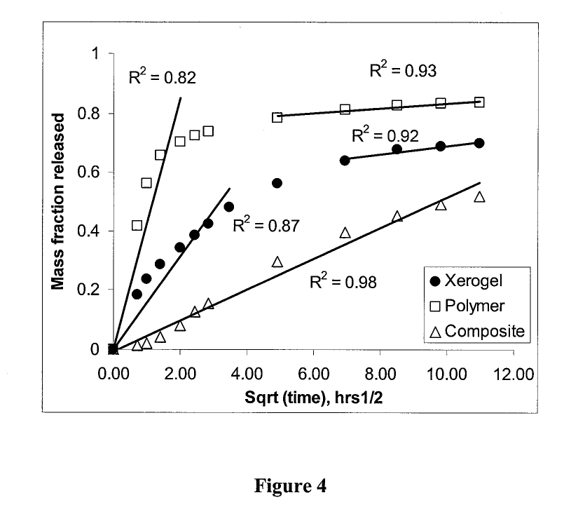

FIG. 4 shows the release profiles from Rₛ15-200 xerogel (16.7% bupivacaine) (represented by solid circles), poly(DTO-20% PEG carbonate) loaded with 8 wt % bupivacaine (represented by squares), and the composite of poly(DTO-20% PEG carbonate) with 50 wt % bupivacaine containing Rₛ15-200 xerogel (represented by triangles). Generally, for both the drug-loaded xerogels and polymer-drug complexes, the bupivacaine release consisted of two stages: an initial faster release, followed by a slower stage. However, when the drug-loaded xerogels are embedded in a suitable polymer matrix to form a composite, the release profile changes significantly, shifting from two-stage release towards a single stage, pseudo first-order release kinetics (release kinetics that are characterized by a substantially constant release of drug over time), as apparent from FIG. 4. The release kinetics of the composite can be tuned by individually adjusting the two components in terms of water:tetraethoxylsilane ratio, pH, drug loading and catalyst used during synthesis (in the case of xerogels), the PEG content, and the length of the pendent ester group (in the case of copolymer).

Thus, composite wound dressings have been prepared from drug-loaded xerogels and tyrosine-derived polycarbonates, and they have been shown a pseudo first-order drug release kinetics over seven days. The release profiles can be tailored as desired by adjusting various parameters for both the xerogels and polymers.

What is claimed:

1. A wound dressing comprising a biocompatible flexible hydrogel comprising biodegradable silica-based microparticles together with at least one therapeutic agent.

2. The wound dressing of claim 1, wherein the flexible hydrogel comprises a tyrosine-based polycarbonate.

3. The wound dressing of claim 1, wherein the biodegradable silica-based microparticles comprise a sol-gel.

4. A wound dressing comprising a biocompatible composite of a flexible polymer film and silica-based ceramic microparticles embedded in the flexible polymer film, wherein at least one of the flexible polymer film and the silica-based ceramic microparticles bind and release at least one therapeutic agent.

5. The wound dressing of claim 4, wherein the therapeutic agent binds to only the silica-based ceramic microparticles.

6. The wound dressing of claim 4, wherein the therapeutic agent binds to both the polymer film and the silica-based ceramic microparticles.

7. The wound dressing of claim 4, wherein the flexible polymer film comprises a tyrosine-based polycarbonate.

8. The wound dressing of claim 4, wherein the silica-based ceramic microparticles comprise a sol-gel.

9. The wound dressing of claim 4, wherein the release of said at least one therapeutic agent is pseudo first-order release.

10. A method of treating a wound comprising applying to the wound a biodegradable biocompatible ceramic-polymeric flexible composite film adapted to provide controlled release of at least one therapeutic agent.

11. The method of claim 10, wherein the therapeutic agent is an anesthetic that functions as a sodium channel blocker to shut down the firing of afferent axons that carry the pain signals back to the brain.

12. The method of claim 10, wherein the therapeutic agent is an anti-apoptotic factor.

13. The method of claim 10, wherein the therapeutic agent is a pro-angiogenic factor.

14. The method of claim 10, wherein the biodegradable biocompatible ceramic-polymeric flexible composite film absorbs extracellular fluid and reduces the hydrostatic pressure and minimizes the extent of damaged tissue.

15. The method of claim 10, wherein said controlled release is pseudo first-order release.
